(19) United States
(12) Patent Application Publication
(10) Pub. No.: US 2007/0099839 A1
(43) Pub. Date: May 3, 2007

(54) Inhibitors of cell migration

(75) Inventors: Michael Stefanidakis, Helsinki (FI); Erkki Koivunen, Helsinki (FI)

Correspondence Address: BIRCH STEWART KOLASCH & BIRCH, PO BOX 747, FALLS CHURCH, VA, 22040-0747, US

(73) Assignee: CIT CANCER TARGETING TECHNOLOGIES, HELSINKI FINLAND (FI)

(21) Appl. No.: 10/561,272

(22) PCT Filed: Jun. 21, 2004
(86) PCT No.: PCT/FI04/00376

§ 371 Date: Dec. 19, 2005

(30) Foreign Application Priority Data
May 4, 2001 (FI) 20010930
Sep. 26, 2001 (FI) 20011882

(51) Int. Cl. A61K 38/08; C07K 7/06

(52) U.S. Cl. 514/17; 530/329

(57) ABSTRACT

The present invention concerns peptide compounds, which were found to bind to the α_{M} integrin I-domain and inhibit its complex formation with proMMP-9, thereby preventing neutrophil migration. The compounds comprise the hexapeptide motif HFDDDE. The compounds can be used in prophylaxis and treatment of inflammatory conditions.

### FIELD OF THE INVENTION

The present invention concerns peptide compounds, which bind to the α_{M} integrin I-domain and inhibit its complex formation with proMMP-9, thereby preventing neutrophil migration. The compounds can be used in treatment of inflammatory conditions.

### BACKGROUND OF THE INVENTION

Polymorphonuclear neutrophils (PMNs) constitute the majority of the blood leukocytes and play a pivotal role in acute inflammation by phagocytosing and killing invading microorganisms. The neutrophils contain four granule compartments: azurophilic granules, specific granules, gelatinase granules, and secretory vesicles, defined by their high content of myeloperoxidase (MO), lactoferrin (LF), gelatinase, and latent alkaline phosphatase, respectively. Proteolytic enzymes, including elastase (1), collagenase (2), and MMP-9 are located in these granules and are important for leukocyte-exit from the bone marrow into the circulation and recruitment into the inflammatory sites (3).

MMP-9 plays a role in tissue remodeling, tissue repair and wound healing, and is a marker of inflammatory diseases such as rheumatoid arthritis (4) and multiple sclerosis (5). PMNs produce MMP-9 during the late stages of maturation in the bone marrow where it is stored in its latent-form (proMMP-9) within the gelatinase granules. Upon cell stimulation, the intracellular granules are rapidly translocated and fused with the plasma membrane. The proMMP-9 zymogen is induced and secreted in response to extracellular stimuli, which initiate specific signalling cascades such as the protein kinase C pathway (6, 7). MMP-9 is also released from human leukocytes after pre-treatment of cells with soluble agonists, such as the complement anaphylatoxin C5a (8) and the tumor necrosis factor-α (INF-α) (9). Cell adhesion to the extracellular matrix is another known stimulus for secretion of proMMP-9 and other MMPs (10, 11). Selective MMP-9 expression is induced as a result of α_{M}β₂ integrin ligation in PMNs (10) and α_{L}β₂ integrin ligation in T lymphoma cells (12).

As a result, three different forms of proMMP-9 are released to the extracellular space as detected by zymography: a 92 kDa monomer, a 200 kDa homodimer, and a 120 kDa complex of MMP-9 bound to neutrophil gelatinase-associated lipocalin (NGAL), a 25 kDa member of the lipocalin family of transport proteins. Activation of proMMP-9 can be achieved extracellularly by proteinases, or chemically by mercurial compounds or reactive oxygen species (13, 14). Once activated, secreted MMP-9 can be inhibited by the tissue inhibitor of metalloproteinases (1) and α₂-macroglobulin present in the extracellular space. However, TIMP only weakly inhibits the surface MMP-9 of neutrophils (15). Thus, the cell surface localization constitutes yet another level for MMP activity regulation.

Recently, we showed that the proMMP-2 and proMMP-9 gelatinases occur in complex with the α_{L}β₂ and α_{M}β₂ integrins on the surface of leukemic cells, when the cells are activated by phorbol ester (16, FI 20030923). The β₂ integrins (CD11/CD18) are pivotal for most leukocyte functions (17, 18). Four 2 integrins have been described: α_{L}β₂ which is predominant in leukocytes, α_{M}β₂ which is enriched in granulocytes and α_{X}β₂ and α_{D}β₂ which are predominantly found in monocytes and macrophages. Their cellular ligands are the intracellular adhesion molecules (ICAMs) 1-5, which are members of the immuno-globulin superfamily. The leukocyte integrins need activation to become fully functional (17). T lymphocytes have been most thoroughly studied and activation can occur through the T cell receptor (17, 19) and may involve protein kinase C (20). In granulocytes, α_{M}β₂ is known to be located intracellularly in specific granules and upon activation it is translocated to the cell surface (21). Not much is known about the mechanism of translocation and which cellular components are involved.

We have mapped the major integrin recognition sequence of proMMP-9 to be present in the MMP catalytic domain (16). That sequence was mimicked by phage display peptides discovered by biopanning on the integrin α_{M} I domain, the most active peptide being ADGACILWMDDGWCGAAG (IDGW). We have studied here the occurrence of the proMMP-9/α_{M}β₂ complex in PMNs and its role in PMN migration. We found that the complex between proMMP-9 and α_{M}β₂ forms already within the gelatinase granules inside the cell and the complex is translocated to the cell surface upon release of the granules during cell activation. Furthermore, a peptide as small as six amino acids in length derived from the MMP-9 catalytic domain was capable of competing with proMMP-9 binding to the β₂ integrin. The hexapeptide and DDGW both attenuated PMN migration in vitro and in vivo, suggesting a role for the MMP-integrin complex in PMN motility.

### SUMMARY OF THE INVENTION

Experiments with recombinant MMP-9 domains gave further support for our finding that a site interacting with the integrin is present on the MMP-9 catalytic domain and we developed an active I domain binding peptide that was only six residues in length. This peptide, HFDDDE, corresponds to a linear sequence from the MMP-9 catalytic domain and efficiently competed with proMMP-9 binding to α_{M}β₂ or its purified I domain. The scrambled peptide had no activity, indicating that the order of the negatively charged amino acids is essential for the activity. Similarly to the phage display-derived DDGW peptide, HFDDDE released cell-bound proMMP-9 and inhibited neutrophil migration in vitro and in vivo. These results suggest that the proMMP-9/α_{M}β₂ complex is important for neutrophil motility but we cannot exclude the possibility that the peptides also affect other β₂ integrin ligands than proMMP-9. However, the fact that DDGW and HFDDDE inhibited the transwell and transendothelial migration of activated neutrophils but not that of resting cells indicates specificity for the action of the peptides. By using CTT, anti-MMP-9 and anti-integrin antibodies, we showed that the peptides inhibited the neutrophil migration that required both proMMP-9 and α_{M}β₂. Similarly as with the THP-1 cell line, we thus find that proMMP-9 is a component of the β₂ integrin-directed neutrophil migration at least under these in vitro conditions.

### DETAILED DESCRIPTION OF THE INVENTION

We have recently demonstrated that promatrix metalloproteinases, particularly proMMP-9, are potent ligands of the leukocyte β₂ integrins. We studied here the complex formation between proMMP-9 and α_{M}β₂, the major MMP and integrin of neutrophils. On resting neutrophils, the proMMP-9/α_{M}β₂ complex was primarily detected in intracellular granules, but after cellular activation it became localized to the cell surface as demonstrated by immunoprecipitation and double immunofluorescence. Further indication of the complex formation was that neutrophils and α_{M}β₂-transfected L cells, but not the wild-type L cells or leukocyte adhesion deficiency (LAD) cells, bound to immobilized proMMP-9 or its recombinant catalytic domain in a β₂ integrin-dependent manner. Peptides that bound to the α_{M} integrin I domain and inhibited its complex formation with proMMP-9 prevented neutrophil migration in a transendothelial assay in vitro and in a thioglycolate-elicited peritonitis in vivo. These results suggest that the translocating proMMP-9/α_{M}β₂ complex may be part of the cell surface machinery guiding neutrophil migration.

In this study, we present evidence that PMNs generate the proMMP-9/α_{M}β₂ complex within their intracellular granules and the complex is translocated to the cell surface when the cells are activated with phorbol ester or via inflammatory mediators. Though proMMP-9 is known to localize to the same intracellular granules as the α_{M}β₂ integrin, association of proMMP-9 with α_{M}β₂ intracellularly has not been shown before. That proMMP-9 is directly able to bind to the α_{M} integrin I domain suggests that the interaction between endogenous proMMP-9 and α_{M}β₂ is direct although we cannot exclude the possibility of accessory molecules. Previously, endogenous neutrophil elastase, proteinase 3, and cathepsin G have all been reported to bind to α_{M}β₂. Thus, α_{M}β₂ may have a specific carrier function for some proteinases. We found that ICAM-1 or fibrinogen do not compete with proMMP-9 binding and the DDGW peptide inhibitor of the proMMP-9/α_{M}β₂ complex is unable to inhibit leukocyte primary adhesion to ICAM-1, fibrinogen or LLG-C4GST but still inhibits the cell migration. These results suggest that α_{M}β₂-bound proMMP-9 is not essential for primary leukocyte adhesion but rather at some other step of cell invasion, perhaps in degradation of the integrin-directed bonds to matrix proteins.

By metabolic labelling of THP-1 leukemic cells, we demonstrated that integrin antibodies coprecipitate proMMP-9 within 2 h after the [³⁵S]-methionine pulse, at the time when the integrin chains are first clearly visible. These results indicate that the proMMP-9 association is an early event for the integrins and that the immunoprecipitated material does not represent endocytosed or recycling integrins. This is in accordance with the double-immunofluorescence studies showing extensive intracellular colocalization of proMMP-9 and α_{M}β₂ in PMNs that have not been subjected to activation. Following PMA-triggered degranulation, we observed dispersion of the staining and the colocalization shifted to the cell surface. Also, the coprecipitation became most intense from the cell surface fraction. These results suggest a rapid translocation of the preformed proMMP-9/α_{M}β₂ complex from the intracellular pool to the cell surface upon activation. This is a more plausible mechanism for the MM/integrin complex formation than binding of a secreted MMP to the integrin on the cell surface. First of all, the integrin could transport the endogenously-bound proMMP-9 to an appropriate site without competition by extracellular MMP inhibitors and integrin ligands. Secondly, as the I domain of α_{M}β₂ does not bind active MMP-9, the integrin could regulate the timing of proMMP-9 activation and release of the active enzyme.

The leukocyte β₂ integrins are involved in leukocyte mobility. Studies with α_{M} or α_{L} knockout mice also show the importance of β₂ integrins in mediating leukocyte adhesive, migratory, and phagocytic activities in response to inflammatory stimuli. Leukocytes from patients with the leukocyte adhesion deficiency syndrome type I (LAD-1) have a defective β₂ integrin subunit and cannot migrate properly although they express proMMP-9, indicating that proMMP-9 alone does not confer cell migration ability. We found that LAD-1 cells expressed MMP-9 immunoreactivity at the leading edge, but did not adhere to the immobilized proMMP-9. Very similar results were obtained with wild type L cells which were unable to adhere to the immobilized proMMP-9 but acquired the ability after transfection of α_{M}β₂. Experiments with PMNs suggest that proMMP-9 would associate with both the intracellular “inactive” integrin and the extracellular integrin once activated by PMA, C5a or TNFα stimulus. It remains to be determined how (pro)MMP-9 is located at the cell surface in LAD-1 cells in the absence of O₂ integrin. There are a number of other binding proteins reported for MMP-9 in the literature.

The cell migration assays revealed two modes of cell motility: β₂ integrin-dependent that was inhibited by DDGW and other peptides, and β₂ integrin-independent that was not inhibited by the peptides. Thus, it is not surprising that the literature is controversial in terms of the role of proMMP-9 in neutrophil migration. Depending on the experimental models and animal species, some studies have supported protease function in neutrophil migration, whereas others have not. The ability of the cells to show different modes of migration with regard to the stimulus could explain many of the discrepancies. The β₂ integrin- and MMP-independent leukocyte migration may correspond to the observed amoeboid-like movement of leukocytes in 3-dimensional collagen under in vitro conditions, which is insensitive to MMP inhibitors.

MMP-9 null mice still show neutrophil migration in thioglycolate-induced peritonitis and in vitro transmigration of neutrophils across TNF-α-treated endothelial cells. However, MMPs are known to have overlapping functions and other MMPs could compensate for the loss of MMP-9. We have previously found that proMMP-2 complexes with α_{M}β₂ and the studies here show that neutrophil MMP-8 can also bind to purified I domain. The HFDDDE sequence is highly conserved in secreted MMPs and such peptides from many MMPs can bind α_{M} I domain in a pepspot membrane assay (16, FI 20030923). It remains to be seen which MMP-integrin complexes are functional in the MMP-9 knockout mice. Furthermore, the ability of α_{M}β₂ to bind also other proteinases such as elastase and urokinase likely affects neutrophil invasivity.

DDGW and HFDDDE had potent activities in vivo in the mouse peritonitis model, but it is unclear to what extent this was due to inhibition of proMMP-9 as both peptides can potentially inhibit other β₂ integrin ligands as well. A subset of β₂ integrin ligands have a DDGW-like sequence and these include, in addition to MMPs, at least complement iC3b and thrombospondin-1. Our results suggest that the proMMP-9/α_{M}β₂ complex may be part of the neutrophil's machinery for a specific β₂ integrin-directed movement.

The present invention is thus directed to new peptide compounds, in specific to a peptide compound comprising the hexapeptide motif HFDDDE. Said compounds can be used as pharmaceuticals, which inhibit neutrophil migration. The inhibitory activity was shown both in in vitro and in vivo experiments. Consequently, the compounds can be used to prevent and treat inflammatory conditions.

The invention thus concerns a compound comprising the hexapeptide motif HFDDDE, and, especially, such a compound for use in inhibiting neutrophil migration, and such a compound for use in prevention and treatment of inflammatory conditions.

The invention is also directed to the compounds of the invention for the manufacture of a pharmaceutical composition for the treatment of conditions dependent on neutrophil migration.

Another embodiment of the invention is a pharmaceutical composition comprising, as an active ingredient a compound of the invention, and a pharmaceutically acceptable carrier.

A further embodiment of the invention is a method for therapeutic or prophylactic treatment of conditions dependent on neutrophil migration, comprising administering to a mammal in need of such treatment a neutrophil migration inhibiting compound of the invention in an amount which is effective in inhibiting migration of neutrophils. Specific embodiments of the invention include methods for prophylaxis and treatment of inflammatory conditions.

Abbreviations: HMEC, human microvascular endothelial cell; PMN, polymorpho-nuclear neutrophil; CTT, CTITHWGFTLC peptide; CTT W→A, CTTHAGFTLC peptide; LLG-C4, CPCFLLGCC peptide; DDGW, ADGACILWMDDGWCGAAG peptide; HSA, human serum albumin; KKGW, ADGACILWMKKGWCGAAG peptide; LF, lactoferrin; MPO, myeloperoxidase; NGAL, neutrophil gelatinase-associated lipocalin; GPA, glycol-phorin A, TAT-2: tumor-associated trypsinogen-2.

### DESCRIPTION OF THE DRAWINGS

FIG. 1A to 1E. Double immunofluorescence staining for α_{M}β₂ and proMMP-9 in human neutrophils and LAD-1 cells. Freshly isolated PMNs (1A, 1B, 1C, and 1D) from healthy donors and LAD-1 cells (1E) were double stained for MMP-9 and α_{M}β₂ integrin (see Experimental). Briefly, unstimulated (1A and 1B) or PMA-stimulated PMNs (1C and 1D) were added to poly-L-lysine-coated coverslips, fixed, and permeabilized (1A and 1C) or not (1B and 1D). Cells were treated with anti-α_{M}β₂ and anti-MMP-9 antibodies followed by staining with FITC-labelled and TRITC-labelled secondary antibodies. (1E) Non-permeabilized LAD-1 cells were fixed and stained similarly. Fluorescence was detected by confocal microscopy (A, B, C D, and E; bars: 4.5, 3.4, 7.0, 4.8, and 5.8 μm, respectively). The experiments were repeated at least 3 times with similar results.

FIG. 2A to 2F. Subcellular fractionation of nitrogen-cavitated disrupted neutrophils on a Percoll gradient. Isolated neutrophils were kept on a resting state or stimulated prior to cell lysis. After Percoll gradient centrifugation, fractions were divided into the populations denoted α, β1, β2, and γ, respectively. S0, supernatant before or after PMA-stimulation; S1, postnuclear supernatant; S2, cytosolic material. These pooled fractions were assayed for MPO (2A), NGAL (2B), LF (2C), MMP-9 (2D), HSA (2E), and HLA (2F) by ELISA. The experiment was repeated at least 3 times with similar results.

FIG. 3A to 3D. Subcellular localization of α_{M}β₂ and MMP-9 in neutrophils granules.

(3A) Equal amounts of total protein from each granule pool (α, β1, β2, and γ-band) were separated by SDS-PAGE and analysed by immunoblotting using polyclonal anti-MMP-9 and the anti-α_{M} antibody MEM170.

(3B) Gelatinase activity from each pool was detected by gelatin zymography. The positions and molecular masses (kDa) of the bands containing gelatinolytic activity are indicated with arrows.

(3C) Solubilized membrane proteins isolated from each pool were immunoprecipitated with the anti-α_{M} antibody OKM10 and detected by western blotting using polyclonal anti-MMP-9 or the anti-α_{M} antibody MEM170.

(3D) THP-1 cells were pulse labeled with [³⁵S]-methionine for 10 min followed by chase for up to 4 h. Cell lysates were incubated with anti-MMP-9, anti-α_{M}, or control (human IgG) antibodies for 3 h. The immunoprecipitates were visualized by fluorography after 24 h. The positions of proMMP-9 and α_{M} subunit are marked.

FIG. 4A to 4D. α_{M}-I domain binding to recombinant MMP-9 domains.

(4A) Schematic representation of MMP-9 and its recombinant forms produced in E. coli.

(4B) ProMMP-9, its recombinant forms or BSA were coated on microtiter wells (80 μg/well) and soluble GST-α_{M} I domain was allowed to bind at the concentrations indicated. The binding was determined by anti-GST monoclonal antibody. The results are means±SD from triplicate wells in this and other figures.

(4C) Binding of proMMP-9 to the immobilized GST-α_{M} I domain was studied in the presence of each peptide at the concentrations indicated. The binding was determined with the anti-MMP-9 antibody GE-213.

(4D) Binding of GST-α_{M} I domain to the immobilised proMMP-8, proMMP-9, ICAM-1, and fibrinogen was studied with ICAM-1, DDGW or KKGW (50 μM) as competitors. In control wells, GST was added instead of GST-α_{M} I domain. The experiment was repeated three times with similar results.

FIG. 5A to 5D. Recognition of recombinant MMP-9 domains by α_{M}β₂ integrin-expressing cells. The studied cells were PMNs (5A, 5B, 5C), α_{M}β₂ L-cell transfectants (5D), non-transfectants (5D), and LAD-1 cells (5D). PMNs were in resting state or stimulated with PMA (5A, 5C) or C5a or TNFα (5B) before the binding experiment to proMMP-9 or its domains. Cells were also pretreated with each peptide (50 μM), antibody (20 μg/ml) or the α_{M} I domain as indicated. Unbound cells were removed by washing and the number of adherent cells was quantitated by a phosphatase assay. The experiment was repeated three times with similar results.

FIG. 6A to 6D. Blockage of PMN and THP-1 cell migration in vitro by gelatinase and β₂ integrin inhibitors. PMNs (1×10⁵ in 100 μl) were applied on the LLG-C4-GST or GST coated surface (6A) or HMEC monolayer (6B) in the absence or presence of peptides (200 μM) or antibodies (20 μg/ml) as indicated. PMNs were stimulated with 20 nM PMA (6A), HMECs with 50 μM C5a or 10 ng/ml TNFα or left untreated (6B). THP-1 cells (5×10⁴ in 100 μl) were stimulated with 50 nM PMA and applied on the coated surfaces together with each peptide (200 μM) (6C). The cells migrated through transwell filters were stained and counted microscopically. All experiments were repeated at least twice. (6D) Phorbol ester-activated THP-1 cells (5×10⁴ in 100 μl) were incubated for 16 h at +37° C. in the presence or absence of peptides as indicated. The conditioned medium was analyzed by gelatin zymography.

FIG. 7A to 7D. Inhibition of neutrophil migration to an inflammatory tissue.

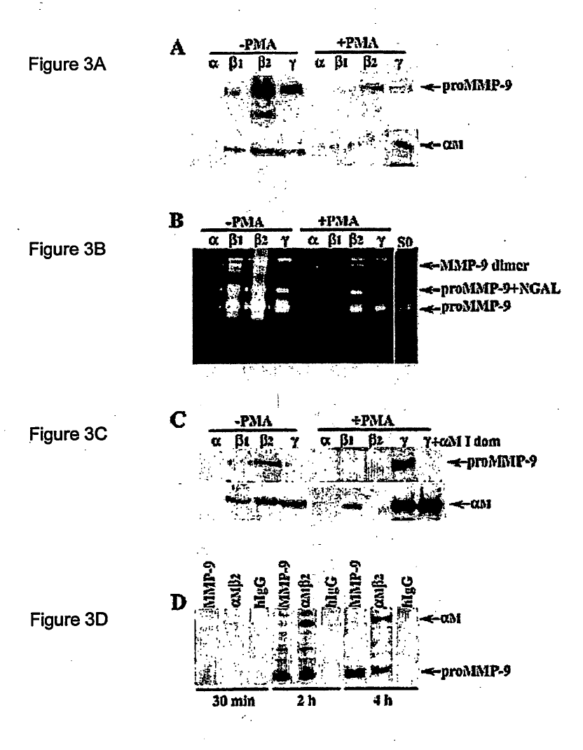

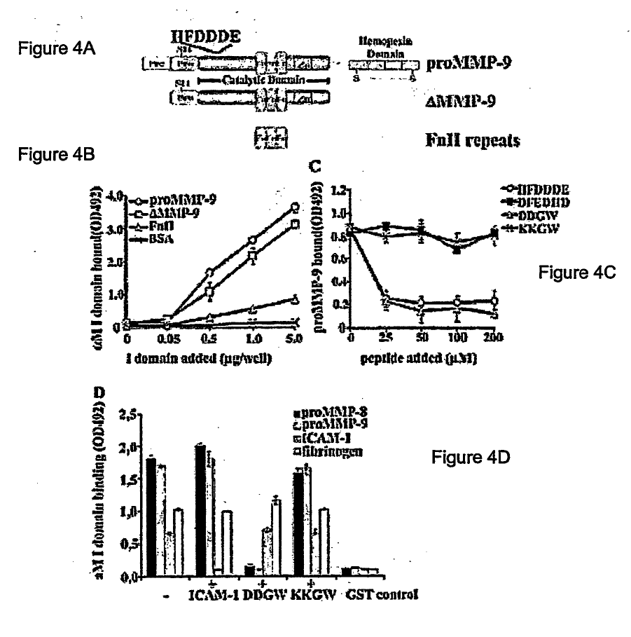

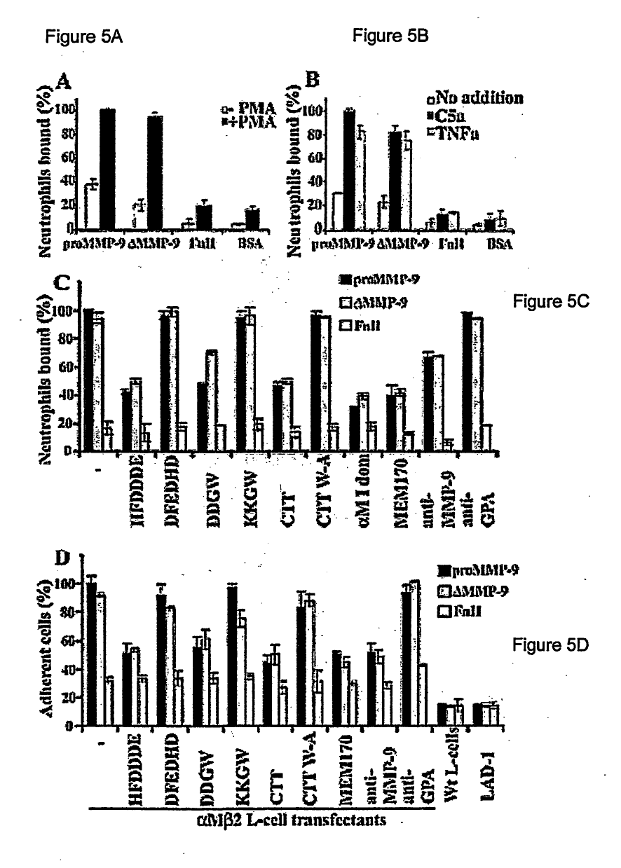

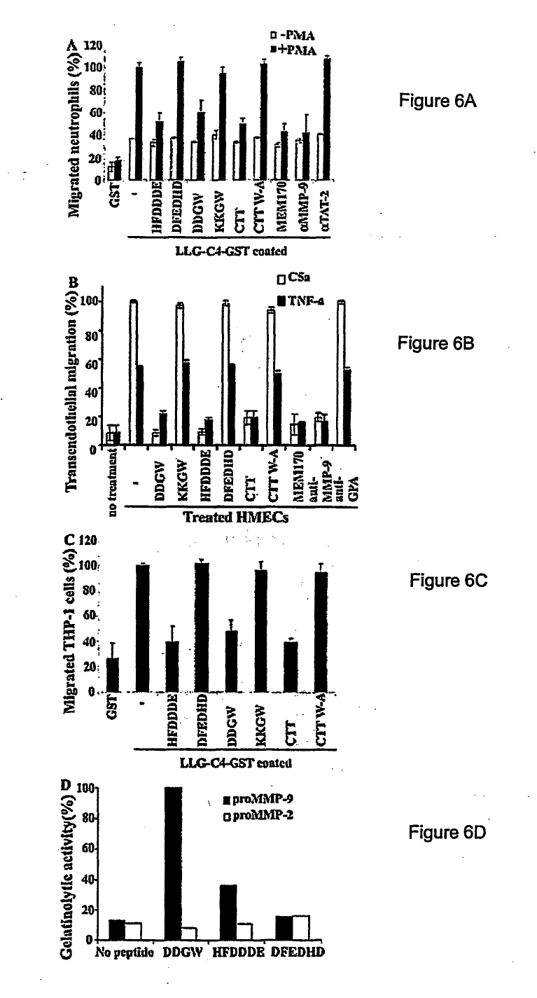

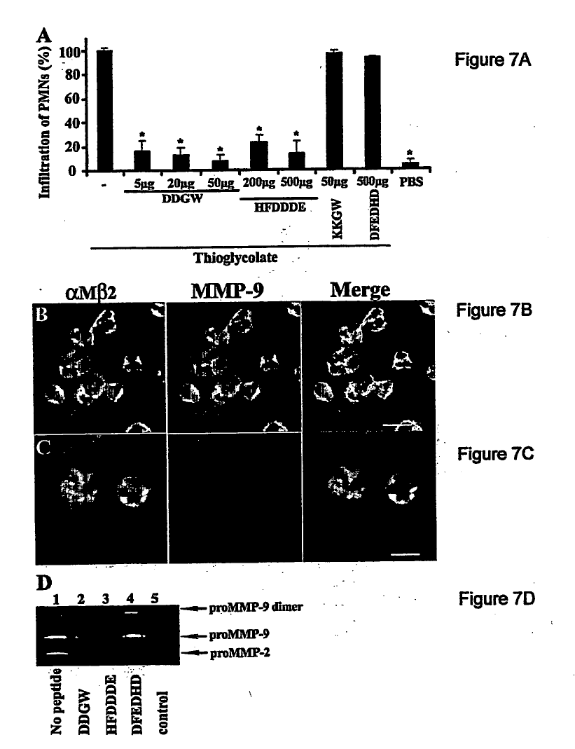

(7A) Mice were injected with thioglycolate or PBS intraperitoneally. The peptides were applied intravenuously at the amounts indicated (A). After 3 h, the intraperitoneal leukocytes were harvested and counted. The results show means±SD of 2-4 mice in a group. (*) indicates statistical significant difference (p<0.001). The experiment was repeated at least 3 times. The infiltrated neutrophils of mice treated with thioglycolate (7B) or PBS (7C) were stained with anti-MMP-9 and anti-α_{M}, as described in the FIG. 3 legend. Fluorescence was studied by confocal microscopy. Bars: 9.1 μm and 4.8 μw, respectively.

(7D) Gelatinolytic activity of the supernatants from the peritoneal cavities of mice collected as in (7A). Lanes 1-4: samples are from thioglycolate-treated mice; lane 5: a sample from PBS-treated mouse. DDGW, HFDDDE, and DFEDHD were injected intravenously at doses of 0.1, 0.2 and 0.2 mg per mouse. The arrows show proMMP-9 dimer, proMMP-9 and proMMP-2. The experiment was repeated three times with similar results.

The publications and other materials referred to or used herein to illuminate the background of the invention, and in particular, to provide additional details with respect to its practice, are incorporated herein by reference. The invention will be described in more detail in the following Experimental Section.

Experimental

Neutrophil Preparations and Cell Lines

PMNs were isolated from peripheral blood anticoagulated in acid-citrate dextrose. Erythrocytes were sedimented by centrifugation on 2% Dextran T-500, and the leukocyte-rich supernatant was pelleted, resuspended in saline and centrifuged on a Lymphoprep (Nyegaard, Oslo, Norway) at 400 g for 30 minutes to separate polymorphonuclear cells from platelets and mononuclear cells (22). PMN purity was >95% with typically <2% eosinophils. Cell viability was measured using an MTT (3-[4,5-dimethylthiazol-2-yl]-2,5-diphenyl tetrazolium) bromide assay as instructed by the manufacturer (Roche).

Human microvascular endothelial cells (HMEC-1) (23), kindly provided by S. Mustoki (Haartman Institute, University of Helsinki), were grown in RPMI 1640 in the presence of 10% FBS containing 2 mM glutamine, 100 IU/ml penicillin and 100 μg/ml streptomycin. Human monocytic THP-1 cells were maintained as described (24, 25). Leukocyte adhesion deficiency type-1 (LAD-1) cells, wild type and α_{M}β₂-transfected L929 mouse fibroblastic cells were generous gifts from Dr. Jean-Pierre Cartron (INSERM, Paris, France). These cells were maintained as described previously (26) and the α_{M}β₂ expression was examined by fluorescence-activated cell sorting (FACS, Becton Dickinson, San Jose, Calif.);

Antibodies and Other Reagents

The monoclonal antibodies MEM170 and OKM10 are against the integrin α_{M} subunit (25). The monoclonal anti-MMP-9 antibody (GE-213) was obtained from LabVision (Fremont, Calif.) and polyclonal MMP-9 from Santa Cruz Biotechnology (Santa Cruz, Calif.). We also used the previously reported affinity purified antibodies against MMP-9 (3). As monoclonal antibody controls, we used a mouse IgG (Silenius, Hawthorn, Australia) and anti-glycophorin A (GPA) (ATCC). Anti-trypsinogen-2 (TAT-2) antibody was a rabbit polyclonal antibody control (27). The peroxidase-conjugated anti-GST mAb was from Santa Cruz Biotechnology. A rat antibody against the mouse α_{M} integrin (MCA74)/and a FITC-conjugated anti-rat (Fab′)₂ were purchased from Serotec (Oxford, UK). The peptides CTT, W→A CTr, LLG-C4, DDGW, and KKGW have been described earlier (16, 28). The BFDDDE and DFEDHD peptides were custom-made by Neosystem (Strasbourg, France). ProMMP-8 and proMMP-9 were obtained from Calbiochem and Roche, respectively. Diisopropyl fluorophosphate was from Aldrich Chemical Company Inc. (Steinheim, Germany). Human C5a and recombinant TNF-α were purchased from Calbiochem (Biosciences, Inc. La Jolla, Calif.) and Sigma-Aldrich (St. Louis, Mo.), respectively.

Subcellular Fractionation

PMNs were suspended in Krebs-Ringer phosphate (130 mM NaCl, 5 mM KCl, 1.27 mM MgSO₄, 0.95 mM CaCl₂, 5 mM glucose, 10 mM NaH₂PO₄/Na₂HPO₄, pH 7.4) at 3×10⁷ cells/ml. PMNs were incubated with or without phorbol myristate acetate (PMA; 2 μg/ml) at +37° C. for 15 minutes, then with 25 mmol/L diisopropyl fluorophosphate for 5 min on ice and the supernatant (S0) was collected. Granule fractions were purified as previously described (29). Briefly, PMNs were disrupted by nitrogen cavitation and cellular debris were removed by centrifugation. The resulting postnuclear supernatant (S1) was applied on a 3-layer Percoll gradient (1.050/1.090/1.120 g/ml) and centrifuged at +4° C. for 30 minutes. Fractions 1 to 6, 7 to 12, 13 to 18, and 19 to 24 (1 ml each) were collected and pooled in 4 distinct groups. The clear cytosol (S2) was present in the last fractions (25 to 30). Aliquots were tested for the presence of marker proteins corresponding to individual compartments (indicated in parenthesis): MPO (a band/azurophil), LF (β1 band/specific), gelatinase (β2 band/gelatinase), albumin (α band/secretory vesicles and plasma membranes) (21). Protein levels were determined using sandwich ELISA assays.

Gelatin Zymography, Immunoprecipitation, and Immunoblotting

Granules fractions were lysed on ice for 15 min with 1% (v/v) Triton-X-100 in phosphate buffered saline (PBS), and the lysate was clarified by centrifugation for 10 min at +4° C. The lysates were analyzed by gelatin zymography on 8% SDS-polyacrylamide gels containing 0.2% gelatin (27). Before immunoprecipitation, the lysate was precleared by incubating for 30 min at +4° C. with protein G-Sepharose. After centrifugation, the supernatant was subjected to immunoprecipitation with polyclonal anti-MMP-9, or monoclonal anti-α_{M} (OKM-10) antibodies. After incubation at +4° C. for 1 h together with protein G-Sepharose, immunocomplexes were pelleted and washed three times with Triton X-100 lysis buffer and once with PBS. Following solubilization in Laemmli sample buffer with 2-mercaptoethanol, the samples were electrophoresed on 4-15% gradient SDS-PAGE gels (Bio-Rad laboratories, Hercules Calif.) and transferred to nitrocellulose membranes (Schleicher & Schuell, Dassel, Germany) by semidry electrophoresis at 15 V for 30 min. Non-specific binding was blocked by soaking the membrane in 5% milk powder in PBS containing 0.05% Tween20 at +4° C. overnight. The membrane was incubated with a monoclonal α_{M} (MEM170) antibody (10 μg/ml) for 2 h at room temperature followed by horseradish peroxidase-conjugated rabbit anti-mouse IgG (1:1000-dilution; DAKO A/S, Copenhagen, Denmark) at 25° C. for 30 min. After several washes, the blot was developed with the Enhanced ChemiLuminescence system (Amersham Pharmacia Biotech) according to the manufacturer's instructions. The membranes were stripped of bound antibodies and reprobed with a polyclonal anti-MMP-9 antibody. An appropriate secondary antibody was used. The membranes were stored in TBS at +4° C. after each immunodetection.

Expression and Purification of Integrin I Domains and MMP-9 Recombinant Proteins

GST-α_{M} and GST-α_{L} I domain fusion proteins were expressed and purified as described previously (30). GST was cleaved from the α_{M} I domain with thrombin (Sigma) and the I domain was purified by ion exchange chromatography on a Mono S HR5/5 column using the FPLC system (Pharmacia). The purification of ΔMMP-9 and FnII domains will be described elsewhere (33). The purity of recombinant proteins was checked by SDS-PAGE.

Interactions Between P-9 Domains and α_{M} I Domain

ICAM-1, fibrinogen, proMMP-8, proMMP-9 or the recombinant domains (0.5 μg/well in PBS) were coated on plastic 96-well plates at +4° C. for 16 h and the wells were blocked with 3% bovine serum albumin (BSA) in PBS for 2 h at room temperature. Binding of the GST-α_{M} I domain was determined essentially as described in the first priority application. In the reverse assay, GST-α_{M} I domain was coated and binding of proMMP-9 was determined using the GE-213 antibody. Competitor peptides were preincubated with the α_{M} I-domain for 20 minutes before the experiment.

Metabolic Radiolabeling and Immunoprecipitation

Non-activated or PMA-activated (50 nM) THP-1 cells (1×10⁷) were subjected to biosynthetic labeling using [³⁵S]-methionine (31). Cells were suspended in methionine-free medium containing 10% dialyzed, heat-inactivated fetal calf serum and were pulsed-labeled with 50 μCi/ml of [³⁵S]-methionine at +37° C. for 10 min. The cells were rapidly washed and further incubated in a complete medium containing 10% FCS at +37° C. for indicated time points. The labeling was stopped by pelleting the cells and adding 2 ml of cold PBS at 3 different time points (30 min, 2 h, and 4 h, respectively). After washings, the cells were lysed with a buffer containing 1% Triton X-100, 10 μg/ml of aprotinin, 10 μg/ml leupeptin, and 1 mM phenylmethylsulfonyl fluoride in PBS, clarified by ultracentrifugation and precleared with protein G-Sepharose. The lysate was immuno-precipitated with affinity purified rabbit anti-MMP-9 and monoclonal α_{M} (OKM-10) (3 μg/ml). A human IgG₁ was a control antibody. After one-hour incubation at +4° C. together with protein G-Sepharose, immunocomplexes were pelleted, washed and run on 7.5% SDS-PAGE gels. The gels were treated with an enhancer (Amplify, Amersham Biosciences), dried on a filter paper and exposed to Kodak X-Omat AR film at −70° C. for a week.

Immunofluorescence Staining

PMNs and LAD-1 cells were treated with 20 nM PMA at ±37° C. for 15 min or left untreated, and then allowed to attach to poly-L-lysine coated cover slips, fixed in 2.5% paraformaldehyde in the presence or absence of 0.1% Triton X-100 at +25° C. for 10 min followed by several washings. The cells were blocked with 20% (v/v) rabbit serum and 3% BSA in PBS at room temperature for 30 min. The cells were incubated with rabbit anti-MMP-9 polyclonal and mouse anti-α_{M} (MEM170) antibodies (1:250 dilution). After washing with PBS, the secondary antibodies, rhodamine (TRITC)-conjugated anti-rabbit or FITC-conjugated anti-mouse (Fab′)₂ (DAKO) were incubated at a 1:500 dilution for 30 min. The samples were mounted and slides were kept in the dark at +4° C. Cellular distribution of α_{M}β₂ and MMP-9 was examined by fluorescence microscopy and confocal microscopy (Leica multi band confocal image spectrophotometer), equipped with 63× magnification oil-immersion objective and a Leica TCS SP2 scan unit.

Cell Adhesion

MMP-9 proteins (200 nM in PBS) were coated at +4° C. for 16 h and the microtiter wells were blocked with 3% BSA in PBS for 1 h at room temperature. The α_{M}β₂-integrin L-cell transfectants and PMNs (1×10⁵ cells/well) were suspended in RPMI medium supplemented with 2 mM MgCl₂ and 0.1% BSA and activated with PMA (20 nM) for 20 min, or with C5a (50 nM) or TNF-α (10 nM) for 4 h at +37° C. The L926 wild type and LAD-1 cells were used as controls. The cells were treated with the indicated antibody (20 μg/ml) or peptide (50 μM) at +37° C. for 30 min, washed twice with serum-free medium and incubated in the microtiter wells at +37° C. for 30 min. The wells were washed with PBS, and the number of adherent cells was quantitated by a phosphatase assay (25).

Cell Migration

Cell migration was conducted using Costar 24-transwell migration chambers with a 3 μm pore size for PMNs and 8 μm for THP-1 cells. To study β₂ integrin-directed migration, the chamber membrane was coated on both sides with LLG-C4-GST integrin ligand (40 μg/ml) or GST as a control and blocked with 10% serum-containing medium (16). To study transendothelial migration, confluent HMECs (4×10⁵ cells/well) were grown on the upper side of the gelatin-coated membrane for 5 days. Culture medium was changed after 3 days. After washing the HMEC layers twice with PBS, chemotactic activation was carried out by adding C5a (50 nM), TNF-α (10 ng/ml), or medium alone to the lower compartment at +37° C. for 4 h. Cultures were then washed again twice to remove all agents. PMNs or THP-1 cells were preincubated with the peptide inhibitor or antibody studied for 1 h before transfer to the upper compartment (1×10⁵ cells in 100 μl RPMI/0.1% BSA or the complete 10% FCS-containing medium). PMNs were allowed to migrate for 2 h through the LLG-C4-GST coated membrane and for 30 min through the HMEC monolayer. THP-1 cells were allowed to migrate for 16 h. The non-migrated cells were removed from the upper surface by a cotton swab and the cells that had traversed the filters were stained with crystal violet and counted.

Mouse Inflammation Model

Balb/c mice at the age of 31-32 weeks were injected intraperitoneally with 3% (w/v) thioglycolate in sterile saline (32). Peptides (5-500 kg in 100 μl) were introduced intravenously through the tail vein. Animals were euthanized after 3 h and the peritoneal cells were harvested by injecting 10 ml of sterile PBS through the peritoneal wall. Red blood cells present in the lavage fluid were removed by hypotonic lysis. Cells were centrifuged and resuspended in 1 ml of sterile 0.25% BSA/Krebs-Ringer. The supernatants were also collected and analysed by gelatin zymography. The number of neutrophils was determined following staining with 0.1% crystal violet and using a light microscope equipped with a ×100 objective. For immunofluorescence staining, cells were allowed to bind to poly-L-lysine coated cover slips, fixed with 2.5% paraformaldehyde in PBS at +4° C. for 30 min followed by several washings. The Fc receptors were blocked in the presence of 20% of rabbit serum and 3% BSA in PBS. The cells were then incubated with anti-MMP-9 polyclonal and α_{M} monoclonal (MCA74) antibodies for 30 min. After washing with PBS, the secondary antibodies, rhodamine (TRITC)-conjugated anti-rabbit or FITC-conjugated anti-rat (Fab′)₂ were incubated for another 30 min. The samples were examined with a confocal microscope. The animal studies were approved by an ethical committee of Helsinki University.

Statistical Analysis

Results were analysed using the F-test (ANOVA) and subsequently, if significant differences between groups occurred, they were subjected to Duncan's Multiple Range test. The program used was SPSS for Windows release 8.0.

Effect of Peptides on Gelatinase Release from Cells

THP-1 cells (50 000/100 μl) were incubated in serum-free RPMI for 16 h in the presence or absence of peptides (200 μM) as described in the text The supernatants from THP-1 cells and mouse intraperitoneal fluid were analysed by gelatin zymography. Gelatinolytic activity was quantified by densitometric scanning.

Results

Intracellular Formation of the proMMP-9/α_{M}β₂ Complex

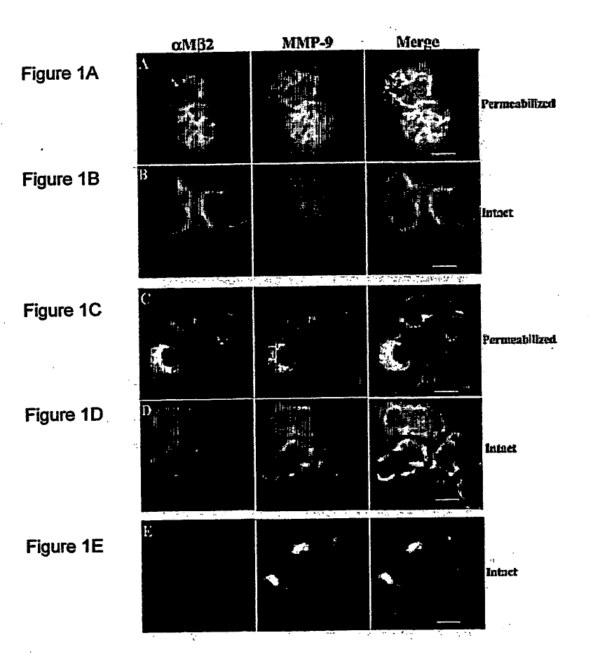

Staining of resting PMNs with am and MMP-9 antibodies showed an intense intracellular colocalization after permeabilization with Triton X-100 (FIG. 1A). In non-permeabilized cells, no such a colocalization was observed and little if any MMP-9 was present on the cell surface (FIG. 1B). After PMA-treatment to cause exocytosis of intracellular granules, the intracellular staining decreased and α_{M}β₂ integrin and MMP-9 colocalized to the cell surface (FIG. 1C-D). Similar results were obtained when exocytosis was triggered with C5a (data not shown). As a control for double immunofluorescence staining, we used a B cell-derived LAD-1 cell line lacking α_{M}β₂ (FIG. 1E). After PMA stimulation, the LAD-1 cells secreted proMMP-9 as detected by zymography analysis and it was expressed on the cell surface but the distribution differed from those of neutrophils, MMP-9 localizing to the leading edge.

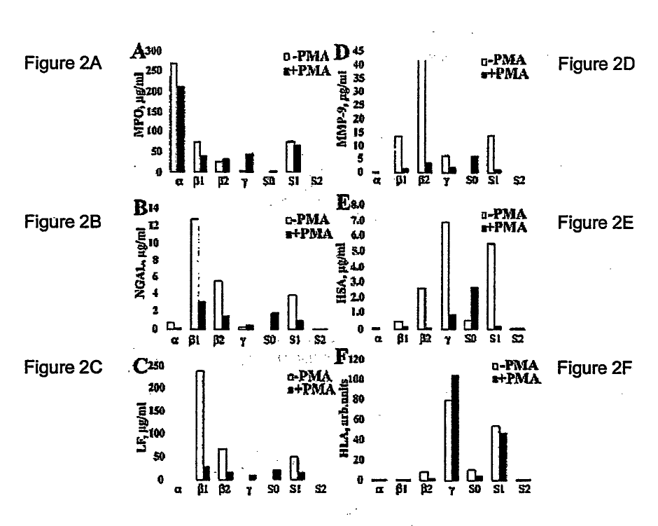

The intracellular colocalization of proMMP-9 and α_{M}β₂ suggested the formation of the proMMP-9/α_{M}β₂ integrin complex in the PMN granules before translocation to the cell surface. We purified the azurophilic (α-band), specific (β1-band), gelatinase (β2-band), and secretory vesicles, including the plasma membranes (γ-band) on a three-step discontinuous Percoll gradient from PMA-treated and non-treated cells. The purity of each fraction was assayed by granule-specific markers. MPO was used as a marker for azurophil granules; LF and NGAL for specific granules; MMP-9 for gelatinase granules; human serum albumin (HSA) for secretory vesicles; and human leukocyte antigen (HLA) for plasma membranes (FIG. 2). PMA induced the release of the majority of the granule markers to the extracellular milieu, whereas MPO was only partially released from azurophil granules. Both NGAL and LF were discharged from the specific granules into the supernatant (S0) by 75% and 90%, respectively. Similarly, the MMP-9 content decreased by 90% in the gelatinase granules and increased in the S0 supernatant. HSA from the secretory vesicles was discharged by 85% and detected in large amounts in S0 supernatant. HLA, a marker of the plasma membrane, remained relatively constant. The levels of HSA, NGAL, LF, and MMP-9 were substantially decreased in the postnuclear supernatants (S1) after cell activation. The cytosolic fraction (S2) was devoid of these markers, indicating that the subcellular fractionation led to the isolation of intact granules.

Immunoblot analysis of the granule fractions showed that proMMP-9 was distributed in the subcellular fractions in a similar way as the α_{M}β₂ integrin (FIG. 3A). In resting PMNs, the major proportion of proMMP-9 and α_{M}β₂ was found in the β2-band, lesser amounts being present in the β1- and γ-bands. In accordance with the immunofluorescence studies, PMA caused a depletion of the β2-band contents. Analysis of the fractions by gelatin zymography similarly showed that PMA decreased the amount of the intracellular proMMP-9 monomer and dimer and its NGAL complex (FIG. 3B). The proMMP-9 zymogen was found in the γ band, representing secretory vesicles and plasma membranes, and in the extracellular milieu (S0).

In the non-activated PMNs, the α_{M} integrin antibody OKM-10 immunoprecipitated the 165 kDa α_{M}-chain from the β1-, β2-, and γ-bands. The 92 kDa proMMP-9 co-precipitated from the β2-band (FIG. 3C). After PMA-stimulation of cells, the α_{M} chain was immunoprecipitated from the β1- and γ-bands but not anymore from the β2-band. The integrin antibody co-precipitated proMMP-9 only from the γ-band. Addition of soluble α_{M} I-domain prevented the co-precipitation.

The biosynthesis of an endogenous complex between proMMP-9 and α_{M}β₂ integrin was investigated in the THP-1 leukemic cell line, which is amenable for such studies. The complex was detected at 2 h and 4 h time points by immunoprecipitation from [³⁵S]-methionine pulsed cells (FIG. 3D, lanes 5 and 8). The OKM10 antibody coprecipitated the α_{M} chain and proMMP-9. The α_{M} chain was only weakly seen in the immunoprecipitates with anti-MMP-9 antibodies (lanes 4 and 7), possibly because of a large excess of unliganded proMMP-9. A control antibody did not coprecipitate α_{M} and proMMP-9 (lanes 3, 6, and 9).

Peptide Inhibitors of the proMMP-9/α_{M}β₂ Complex Prevent Neutrophil Migration

In our previous study, pepspot analysis located the integrin interactive site of proMMP-9 to a 20-amino acid long sequence present in the catalytic domain, QGDAHFDDDELWSLGKGVVV (see the first priority document). Further screening by the pepspot system has indicated that sufficient integrin binding activity is achieved by truncating this sequence to a hexapeptide, HFDDDE (data not shown). To confirm that such a short sequence is the bioactive site of proMMP-9, we first prepared bacterially expressed recombinant domains of MMP-9 (FIG. 4A). ΔMMP-9 is composed of the prodomain (Pro) and the catalytic domain but lacks the hemopexin domain The fibronectin type II repeats (FnII) were also produced as a separate recombinant protein as this is an important substrate-binding region. The procatalytic domain construct ΔMMP-9 bound the α_{M} I domain nearly as efficiently as the wild type proMMP-9 (FIG. 4B). FnII protein almost lacked activity. The HFDDDE peptide identified by the solid-phase pepspot analysis was highly active when made by peptide synthesis and inhibited proMMP-9 binding to the α_{M} I domain with an IC₅₀ of 20 μM (FIG. 4C). The bound proMMP-9 was determined with the GE-213 antibody, which recognizes an epitope of the FnII domain (data not shown). A scrambled peptide DFEDHD with the same set of negatively charged amino acids was inactive. HFDDDE was equally potent as DDGW, the α_{M} I domain-binding peptide discovered by phage display. KKGW, the control peptide for DDGW, was without effect. As the HFDDDE sequence is highly conserved in the members of the MMP family, we also examined the α_{M} I domain binding to human neutrophil collagenase, MMP-8. I domain showed a similar DDGW-inhibitable binding to proMMP-8 as to proMMP-9 (FIG. 4D). ICAM-1 and fibrinogen did not compete with either proMMP, implying different binding sites for the matrix proteins and proMMPs in the I domain.

After integrin activation, PMNs exhibited an ability to adhere on proMMP-9. PMA-stimulated PMNs bound to microtiter well-coated ΔMMP-9 nearly as strongly as to proMMP-9 (FIG. 5A). Stimulation of PMNs with C5a or TNF-α gave similar results PMN adherence increasing by 3-fold (FIG. 5B). The FnII domain did not support PMN adhesion. PMN adherence was inhibited by HFDDDE (50 μM), DDGW (50 μM), the soluble α_{M} I domain and the MEM170 antibody (FIG. 5C), indicating β₂ integrin-directed binding. The control peptides (DFEDHD, KKGW) and an irrelevant monoclonal antibody (anti-GPA) had no effect. The CTT peptide, but not the W→A CTT control peptide lacking MMP inhibitory activity, binds to the MMP-9 catalytic domain (unpublished results) and also inhibited the PMN adherence. MMP-9 antibodies inhibited partially.

We, also examined α_{M}β₂-transfected L cells. The α_{M}β₂ L-cell transfectants bound to proMMP-9 and ΔMMP-9 similarly as PMNs did and the I domain ligands and MMP-9 inhibitors attenuated the binding (FIG. 5D). The transfected cells also showed a weak adherence to FnII domain, but the studied peptides and antibodies did not inhibit this binding. Wild type L cells or LAD-1 cells showed no binding to proMMP-9 or its domains.

The in vitro migration of PMNs was studied on transwell filter assays. Coating with the artificial β₂ integrin ligand LLG-C4-GST renders cell migration dependent on the β₂ integrins (16, 25). The migration of PMA-activated PMNs was 5-fold in the LLG-C4-GST substratum in comparison to GST substratum (FIG. 6A). HFDDDE (200 μM) inhibited the migration of PMA-stimulated cells but not the basal migration of non-activated cells. DDGW, CTT, MEM170 (20 μg/ml) and polyclonal anti-MMP-9 (20 μg/ml) worked similarly, affecting the migration of the PMA-activated cells only. Control peptides and an antibody control (anti-TAT-2) had no effect Similar results were obtained in a transendothelial migration assay (FIG. 6B). Chemotaxis with C5a or TNF-α increased PMN transmigration by 5-10 fold and inhibition was obtained by DDGW, HFDDDE, and CTT but not with the control peptides. Similarly, α_{M} and MMP-9 antibodies inhibited but an antibody control (anti-GPA) did not. We also examined the effects of peptides on THP-1 leukemia cell migration through the LLG-C4GST coated transwell filters. The results were the same as for PMNs. HFDDDE, DDGW, and CTT inhibited THP-1 migration and the control peptides did not (FIG. 6C).

Previous studies with the DDGW peptide showed that it can release proMMP-9 from THP-1 cells (FI 2003 0923). We found that the HFDDDE peptide also released proMMP-9 but was less effective than DDGW (FIG. 6D). The scrambled peptide did not induce the release of proMMP-9. Under the 16 h incubation time, the peptides had no effect on the secretion of proMMP-2.

To study neutrophil migration in vivo, we used a mouse model of thioglycolate-induced peritonitis. The cells that infiltrated into the peritoneal cavity within 3 h after thioglycolate irritant were judged to be predominantly PMNs by crystal violet staining. The DDGW and HFDDDE peptides had potent in vivo activities in this inflammation model (FIG. 7A). An intravenous tail injection of DDGW or HFDDDE inhibited the intraperitoneal accumulation of PMNs. The KKGW and DFEDHD peptides used as controls had no effect. The effects of DDGW and HFDDDE were concentration-dependent and up to 90% inhibition was obtained by doses of 50 μg and 500 μg per mouse, respectively. DDGW was active even at 5 μg given per mouse corresponding to an effective dose of 0.1 mg/kg mouse tissue. Approximately 20-fold more PMNs were present intraperitoneally after thioglycolate-stimulus in comparison to the PBS control. The collected inflammatory PMNs stained positively for the proMMP-9/α_{M}β₂ complex by double immunofluorescence (FIG. 7B). The cells collected after PBS injection lacked the complex; they expressed the integrin but had no cell-surface MMP-9 (FIG. 7C). Zymography analysis of the supernatants from the collected intraperitoneal fluid showed that thioglycolate induced elevated levels of gelatinases in comparison to PBS (FIG. 7D). DDGW and HFDDDE, but not the scrambled peptide, prevented the increase in gelatinase levels in accordance with the inhibition of cell migration. I

### REFERENCES

1. Dewald, B., R Rindlerludwig, U. Bretz, and M. Baggiolini. 1975. Subcellular-localization and heterogeneity of neutral proteases in neutrophilic polymorphonuclear leukocytes. J. Exp. Med 141:709.
2. Murphy, G., U. Bretz, M. Baggiolini, and J. J. Reynolds. 1980. The latent collagenase and gelatinase of human polymorphonuclear neutrophil leukocytes. Biochem. J. 192:517.
3. Kjeldsen, L., O. W. Bjerrum, J. Askaa, and N. Borregaard. 1992. Subcellular-localization and release of human neutrophil gelatinase, confi rig the existence of separate gelatinase-containing granules. Biochem. J. 287:603.
4. Opdenakker, G., P. E. van den Steen, and J. van Damme. 2001. Gelatinase B: a tuner and amplifier of immune functions. Trends Immunol. 22:571.
5. Gijbels, K., S. Masure, H. Carton, and G. Opdenakker. 1992. Gelatinase in the cerebrospinal-fluid of patients with multiple-sclerosis and other inflammatory neurological disorders. J. Neuroimmunol. 41:29.
6. Xie, B., A. Laouar, and E. Huberman. 1998. Fibronectin-mediated cell adhesion is required for induction of 92-kDa type IV collagenase/gelatinase (MMP-9) gene expression during macrophage differentiation—The signaling role of protein kinase C-beta. J. Biol. Chem. 273:11576.
7. Liu, J. F., M. Crepin, J. M. Liu, D. Barritault, and D. Ledoux. 2002. FGF-2 and TPA induce matrix metalloproteinase-9 secretion in MCF-7 cells through PKC activation of the Ras/ERK pathway. Biochem. Biophys. Res. Commun. 293:1174.
8. Takafuji, S., A. Ishida, Y. Miyakuni, and T. Nakagawa 2003. Matrix metalloproteinase-9 release from human leukocytes. J. Invest. Allerg. Clin. Immunol. 13:50.
9. Robinson, S. C., K. A. Scott, and F. R. Balkwill. 2002. Chemokine stimulation of monocyte matrix metalloproteinase-9 requires endogenous TNF-alpha. Eur. J. Immunol. 32:404.
10. Wize, J., I. Sopata, A. Smerdel, and S. Maslinski. 1998. Ligation of selectin L and integrin CD11b/CD18 (Mac-1) induces release of gelatinase B (MMP-9) from human neutrophils. Inflamm. Res. 47:325.
11. Galt, S. W., S. Lindemann, D. Medd, L. L. Allen, L. W. Kraiss, E. S. Harris, S. M. Prescott, T. M. McIntyre, A. S. Weyrich, and G. A. Zimmerman. 2001. Differential regulation of matrix metalloproteinase-9 by monocytes adherent to collagen and platelets. Circ. Res. 89:509.
12. Aoudjit, F., E. F. Potworowski, and Y. St-Pierre. 1998. Bi-directional induction of matrix metalloproteinase-9 and tissue inhibitor of matrix metalloproteinase-1 during T lymphoma endothelial cell contact: Implication of ICAM-1. J. Immunol. 160:2967.
13. Mazzieri, R., L. Masiero, L. Zanetta, S. Monea, M. Onisto, S. Garbisa, and P. Mignatti. 1997. Control of type IV collagenase activity by components of the urokinase-plasmin system: A regulatory mechanism with cell-bound reactants. EMBO J. 16:2319.
14. Ramos-DeSimone, N., E. Hahn-Dantona, J. Sipley, H. Nagase, D. L. French, and J. P. Quigley. 1999. Activation of matrix metalloproteinase-9 (MMP-9) via a converging plasmin/stromelysin-1 cascade enhances tumor cell invasion. J. Biol. Chem. 274:13066.
15. Owen, C. A., Z. M. Hu, B. Barrick, and S. D. Shapiro. 2003. Inducible expression of TIMP-resistant matrix metalloproteinase-9 on the cell surface of neutrophils. Am. J. Respir. Cell Mol. Biol. 29:283.
16. Stefanidakis, M., M. Bjbrklund, E. Ihanus, C. G. Gahmberg, and E. Koivunen. 2003. Identification of a negatively charged peptide motif within the catalytic domain of progelatinases that mediates binding to leukocyte β2 integrins. J. Biol. Chem. 278:34674.
17. Dustin, M. L., and T. A. Springer. 1989. T-cell receptor cross-linking transiently stimulates adhesiveness through LFA-1. Nature 341:619.
18. Gahmberg, C. G. 1997. Leukocyte adhesion: CD11/CD18 integrins and intercellular adhesion molecules. Curr. Opin. Cell Biol. 9:643.
19. Springer, T. A. 1990. Adhesion receptors of the immune-system. Nature 346:425.
20. Fagerholm, S., T. J. Hilden, and C. G. Gahmberg. 2002. Lck tyrosine kinase is important for activation of the CD11a/CD18-integrns in human T lymphocytes. Eur. J. Immunol. 32:1670.
21. Sengelov, H., P. Follin, L. Kjeldsen, K. Lollike, C. Dahlgren, and N. Borregaard. 1995. Mobilization of granules and secretory vesicles during in-vivo exudation of human neutrophils. J. Immunol. 154:4157.
22. Boyum, A. 1968. Isolation of mononuclear cells and granulocytes from human blood. Isolation of mononuclear cells by one centrifugation and of granulocytes by combining centrifugation and sedimentation at lg. Scand. J. Clin. Lab Invest. 97:77.
23. Mustjoki, S., R. Alitalo, E. Elonen, O. Carpen, C. G. Gahmberg, and A. Vaheri. 2001. Intercellular adhesion molecule-1 in extravasation of normal mononuclear and leukaemnia cells. Br. J. Haematol. 113:989.
24. Koivunen, E., W. Arap, H. Valtanen, A. Rainisalo, O. P. Medina, P. Heikkila, C. Kantor, C. G. Gahmberg, T. Salo, Y. T. Konttinen, T. Sorsa, E. Ruoslahti, and R Pasqualini. 1999. Tumor targeting with a selective gelatinase inhibitor. Nat. Biotechnol. 177:768.
25. Koivunen, E., T. M. Ranta, A. Annila, S. Taube, A. Uppala, M. Jokinen, G. van Willigen, E. Ihanus, and C. G. Gahmberg. 2001. Inhibition of beta(2) integrin-mediated leukocyte cell adhesion by leucine-leucine-glycine motif-containing peptides. J. Cell Biol. 153:905.
26. Hermand, P., M. Huet, I. Callebaut, P. Gane, E. Ihanus, C. G. Gahmberg, J. P. Cartron, and P. Bailly. 2000. Binding sites of leukocyte beta(2) integrins (LFA-1, Mac-1) on the human ICAM-4/LW blood group protein. J. Biol. Chem 275:26002.
27. Sorsa, T., T. Salo, E. Koivunen, J. Tyynela, Y. T. Konttinen, U. Berginann, A. Tuuttila, E. Niemi, O. Teronen, P. Heikkila, H. Tschesche, J. Leinonen, S. Osman, and U. H. Stenman. 1997. Activation of type IV procollagenases by human tumor-associated tyypsin-2. J. Biol. Chem. 272:21067.
28. Bjorklund, M., H. Valtanen, H. Savilahti, and E. Koivunen. 2003. Use of intein-directed peptide biosynthesis to improve serum stability and bioactivity of a gelatinase inhibitory peptide. Comb. Chem. High Throughput Screen 6:29.
29. Kjeldsen, L., H. Sengelov, and N. Borregaard. 1999. Subcellular fractionation of human neutrophils on Percoll density gradients. J. Immunol. Methods 232:131.
30. hanus, E., L. Uotila, A. Toivanen, M. Stefanidakis, P. Bailly, J. P. Cartron, and C. G. Gahmberg. 2003. Characterization of ICAM-4 binding to the I domains of the CD11a/CD18 and CD11b/CD18 leukocyte integrins. Eur. J. Biochem. 270:1710.
31. Jokinen, M., C. G. Gahmberg, and L. C. Andersson. 1979. Biosynthesis of the major human red-cell sialoglycoprotein, glycophorin-A, in a continuous cell-line. Nature 279:604.
32. Ajuebor, M. N., A. M. Das, L. Virag, R. J. Flower, C. Szabo, and M. Perretti. 1999. Role of resident peritoneal macrophages and mast cells in chemokine production and neutrophil migration in acute inflammation: Evidence for an inhibitory loop involving endogenous IL-10. J. Immunol. 162:1685.
33. Submitted manuscript: Mikael Björklund, Pia Heikkilä and Erkki Koivunen. Title: Peptide inhibition of catalytic and noncatalytic activities of matrix metalloproteinase-9 blocks tumor cell migration and invasion.

1. A compound comprising the hexapeptide motif HFDDDE.

2. The compound according to claim 1 for use as a pharmaceutical.

3. The compound according to claim 1 for use in inhibiting neutrophil migration.

4. The compound according to claim 1 for use in prevention and treatment of inflammatory conditions.

5. A pharmaceutical composition comprising the compound according to claim 1, and a pharmaceutically acceptable carrier.

6. Use of the compound according to claim 1 for the manufacture of a pharmaceutical composition for prophylaxis and treatment of conditions dependent on neutrophil migration.

7. Use of the compound according to claim 1 for the manufacture of a pharmaceutical composition for prophylaxis and treatment of inflammatory conditions.

8. A method for therapeutic or prophylactic treatment of conditions dependent on neutrophil migration, comprising administering to a mammal in need of such treatment a compound comprising the hexapeptide motif HFDDDE in an amount which is effective in inhibiting neutrophil migration.

9. A method for therapeutic or prophylactic treatment of inflammatory conditions, comprising administering to a mammal in need of such treatment a compound comprising the hexapeptide motif HFDDDE in an amount which is effective in inhibiting neutrophil migration.
